(19) United States
(12) Patent Application Publication
(10) Pub. No.: US 2009/0228003 A1
(43) Pub. Date: Sep. 10, 2009

(54) Tissue ablation device using radiofrequency and high intensity focused ultrasound

(75) Inventor: Yegor Sinelnikov, Port Jefferson, NY (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: ProRhythm, Inc., Ronkonkoma, NY (US)

(21) Appl. No.: 12/074,559

(22) Filed: Mar. 4, 2008

(51) Int. Cl. A61B 18/14

(52) U.S. Cl. 606/41

(57) ABSTRACT

Apparatus and methods for ablating tissue such as cardiac tissue. The apparatus includes a probe carrying a first ablation element 11 which may include an ultrasonic transducer and a balloon structure which directs the ultrasonic energy, and an additional ablation element 17 located distal to the first ablation element. The mode of operation of the additional ablation element 17 may be different from that of the first ablation element 20. Both ablation elements may be positioned by positioning the probe. The first ablation element may be arranged to form a loop-like lesion, whereas the additional ablation element may be arranged to form a spot-like lesion.

### FIELD OF THE INVENTION

The present application relates to devices and medical procedures for ablating tissue, more particularly to devices and procedures for ablating heart tissue.

### BACKGROUND OF THE INVENTION

In certain medical procedures, it is desirable to heat tissue surrounding an anatomical structure such as a blood vessel or a gastrointestinal, urinary, genital, or respiratory structure. Depending upon the condition to be treated, energy may be applied to the tissue constituting the wall of the structure, or to tissue surrounding the wall. Energy may be applied to heat the tissue to a degree sufficient to cause death of the tissue. Heating to this degree is referred to herein as “ablation.” Typically, heating to about 60-80° C. for a short time is sufficient.

Ablation of tissue in patients with atrial fibrillation or “AF” has been proposed heretofore. Contraction or “beating” of the heart is controlled by electrical impulses generated at nodes within the heart and transmitted along conductive pathways extending within the wall of the heart. Certain diseases of the heart known as cardiac arrhythmias involve abnormal generation or conduction of the electrical impulses. One such arrhythmia is atrial fibrillation. Certain cardiac arrhythmias can be treated by deliberately damaging the tissue along a path crossing a route of abnormal conduction. This causes formation of a scar extending along the path where disruption occurred. The scar blocks conduction of the electrical impulses. The abnormal electrical impulses can be carried by abnormal structures extending within the wall of a pulmonary vein. Conduction of these abnormal electrical impulses may be blocked by forming a scar in the wall of the pulmonary vein or in the opening or ostium of the pulmonary vein. For example, as described in U.S. Pat. No. 5,575,766, ablation may be performed using a catheter having an ablation element such as an RF electrode at its tip. The physician maneuvers the catheter so that the tip moves along the heart wall while the electrode is active to trace the desired scar on the heart wall. This approach manifestly requires a difficult series of manipulations by the physician. U.S. Pat. No. 5,971,983 depicts an elongated ablation catheter having numerous ablation elements, as for example, RF electrodes, arranged along its length so that, at least in theory, an elongated lesion can be formed by positioning the catheter against an elongated region of the heart wall and actuating the ablation elements. U.S. Pat. No. 6,254,599 recites an ablation device carried on the tip of a catheter and adapted for insertion into a pulmonary vein. The ablation device is assertedly capable of forming a ring-like lesion encircling the vein. Certain embodiments of the '599 patent show such a ring-forming device mounted at the distal end of an elongated catheter with numerous additional ablation elements arrayed along its length so that a linear lesion can be formed in conjunction with the ring-like region.

Commonly assigned U.S. Pat. No. 6,635,054, the disclosure of which is incorporated by reference herein, teaches, inter alia, an ablation device using an ultrasonic emitter and a reflector formed by a balloon structure to focus ultrasonic energy from the emitter into a ring-like focal region. As discussed in the '054 patent, such a device can be used to form a ring-like lesion in the heart wall, encircling the ostium of a pulmonary vein. Commonly assigned U.S. Patent Publication No. 2004/0176757 discloses, inter alia, a similar ablation device which is mounted on a steerable catheter. As taught in the '757 publication, such a steerable balloon device can be positioned in the desired relationship to the heart wall readily, even where the pulmonary veins lie at unusual angles to the heart wall or have other irregular features. As also taught in certain embodiments of the '757 publication, the steerable ablation device can be used to form linear or spot lesions by turning the device to lie at an appropriate orientation relative to the heart wall. The preferred apparatus and methods in accordance with the '054 patent and '757 publication can provide effective therapy for arrhythmias such as AF. However, still further improvement would be desirable.

### SUMMARY OF THE INVENTION

An ablation device according to one aspect of the present invention includes a catheter having a first ablation element secured to the catheter. A second ablation element is secured to the catheter distal to the first ablation element. The second ablation element's mode of operation is different from the first ablation element. For example, the first ablation element may be an ultrasonic ablation element, whereas the second ablation element may be an electrode for application of RF or other electrical energy. The catheter may be steered to position at least one of the first ablation element or the second ablation element in a desired location relative to a tissue to be ablated.

One aspect of the invention provides apparatus for cardiac treatment. The apparatus according to this aspect of the invention desirably includes a probe having proximal and distal ends and a first ablation element secured to the probe at or adjacent the distal end thereof. The first ablation element may include an expansible balloon structure and an ultrasonic transducer mounted within the balloon structure, the balloon structure having a distal end and a proximal end, the ultrasonic transducer and balloon structure being constructed and arranged so that ultrasonic energy emitted by the ultrasonic transducer will be directed through the balloon structure. The apparatus according to this aspect of the invention most preferably also includes an additional ablation element secured to the probe and located distal to the ultrasonic transducer and at least partially outside the balloon structure. The first ablation element may be arranged to form an arcuate or loop-like lesion, whereas the second ablation element may be arranged to form a spot lesion.

A further aspect of the invention provides methods of ablating cardiac tissue to impede flow of abnormal electrical signals. A method according to this aspect of the invention desirably includes the steps of: inserting an elongated probe so that a distal end of the probe and an first ablation element carried on the probe is disposed in a chamber of the heart, ablating tissue using the first ablation element to form a lesion, positioning an additional ablation element by steering the probe, and ablating tissue using the additional ablation element. For example, the first ablation element may be used to form a loop-like lesion, and the additional ablation element may be used to ablate spots at gaps in the loop-like lesion, to form linear lesions, or both.

Other objects, features and advantages of the present invention will be more readily apparent from the detailed description of the preferred embodiments set forth below, taken in conjunction with the accompanying drawings.

### BRIEF DESCRIPTION OF THE DRAWINGS

FIG. 1 is a schematic sectional view depicting an ablation device according to an embodiment of the invention in conjunction with cardiac structures.

FIG. 2 is a fragmentary schematic view of depicting a portion of the ablation device of FIG. 1 with certain elements omitted for clarity of illustration.

FIG. 3 is view similar to FIG. 2, but depicting the ablation device in a different stage of operation.

FIG. 4 is a view similar to FIG. 2 depicting a portion of an ablation device according to a still further embodiment of the invention.

FIG. 5 is a schematic view depicting an ablation device according to yet another embodiment of the invention.

### DETAILED DESCRIPTION

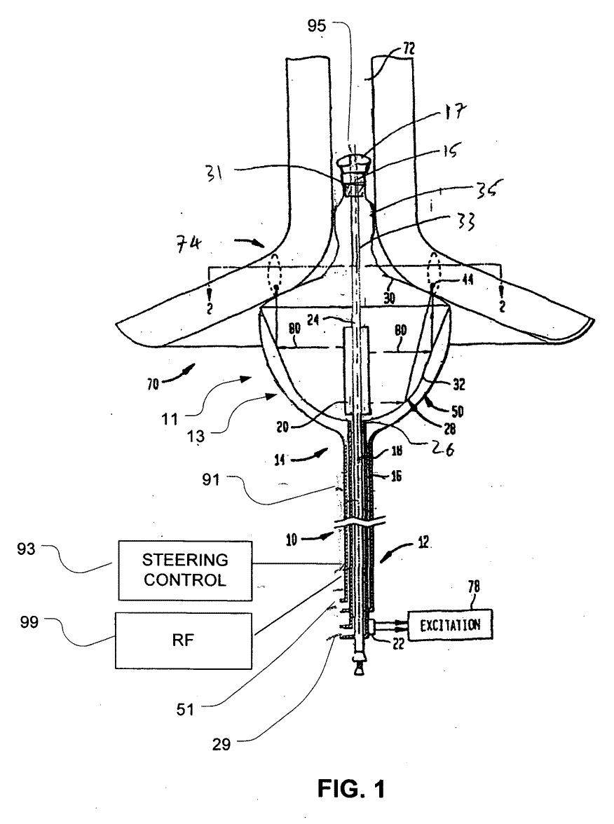

FIG. 1 shows one exemplary embodiment of the ablation device of the invention. As used in this disclosure with reference to structures which are advanced into the body of a subject, the “distal” end of such a structure should be taken as the end which is inserted first into the body and which penetrates to the greatest depth within the body, whereas the proximal end is the end of the structure opposite to the distal end.

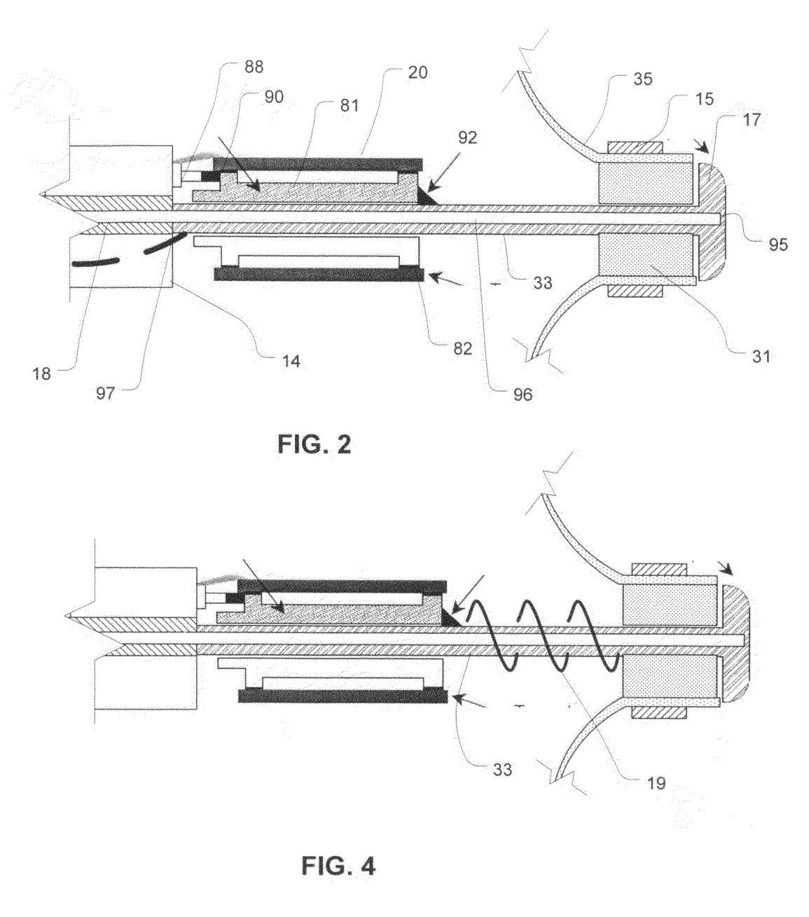

The ablation device of FIGS. 1-3 includes a first ablation element 11 which incorporates an inflatable balloon structure 13 and an ultrasonic transducer 20 disposed within the balloon structure 13. As best seen in FIG. 2, the first ablation element 11 is mounted at the distal end 14 of an elongated probe 10. The probe structure also has a proximal end 12. A portion of a probe structure 10 between the proximal and distal ends is omitted in FIG. 2 for clarity of illustration. The probe structure is includes a first catheter 16 defining a plurality of lumens including a lumen 18.

Transducer 20 is generally in the form of a hollow, cylindrical tube of piezoelectric material having electrically conductive layers (not shown) on its interior and exterior surfaces. As best seen in FIG. 2, a generally tubular strain relief barrel 81 is mounted on the distal end of catheter 16. Barrel 81 may be made from brass or any other suitable material. Barrel 81 has projections 82 and 84 at its distal and proximal ends. The surfaces of projections 82 and 84 form a surface for mounting transducer 20. The conductive coating on the outer surface 86 of transducer 20 is electrically connected to the shield of a HIFU coaxial cable 88 which extends within a lumen of the catheter 16 and which is connected to a source 78 of electrical excitation signals through a connector 22 at or near the proximal end of the probe. The central conductor 90 of coaxial cable 88 is also connected to source 78 of electrical excitation signals through connector 22. The central conductor 90 is electrically connected to barrel 81 and thus electrically connected to the coating on the inside surface of transducer 20.

The first catheter 16 and transducer 20 define a central axis 24 adjacent the distal end of the probe structure. A first balloon 28, also referred to herein as a “structural balloon” is mounted to the distal end of catheter 16, and communicates with a first inflation port 29 near the proximal end of the probe. First balloon 28 includes an active wall 32 formed from a film which is flexible but which can form a substantially noncompliant balloon structure when inflated. The first balloon also includes a forward wall 30, which may be generally conical or dome-shaped and may project forwardly from its juncture with active wall 32. Active wall 32 joins the wall of catheter 16 proximally of transducer 20. Thus, transducer 20 is disposed inside of first balloon 28.

A second balloon 50, also referred to herein as the “reflector balloon,” is carried on the distal end of catheter 16, and communicates with a second inflation port 51 adjacent the proximal end of the catheter. The interior spaces within the first balloon 28 and second balloon 50 do not communicate with one another. The active wall 32 of the first balloon also serves as a wall of the second balloon. When both first and second balloons 28 and 50, respectively, are in a deflated position, second balloon 50 is collapsed inwardly, toward central axis 24 so that second balloon 50 in a deflated condition closely overlies deflated first balloon. In the inflated, operative condition depicted in FIG. 2, the first balloon 28 is filled with a liquid, as for example, an aqueous liquid such as saline solution, whereas the second balloon 50 is filled with a gas such as carbon dioxide. Because of the difference in acoustic impedance between the liquid in the first balloon 28 and the gas in second balloon 50, the boundary between the first and second balloons, at active wall 30, is highly reflective to ultrasound. The catheter 16 and the mounting of the transducer 20 within the catheter may be constructed and arranged so that a liquid can be circulated into and out of the balloon while balloon 28 is maintained inflated by the liquid, and so that the circulating liquid passes over transducer 20 to cool it.

As discussed above, transducer 20 is connected to a source 78 of electrical excitation signals through connector 22. Source 78 is adapted to provide electrical excitation. Thus, source 78 can provide continuous excitation for a predetermined period of time and then turn the electrical excitation off for a predetermined period of time. The electrical excitation may be turned on and off as required. The electrical excitation actuates transducer 20 to produce ultrasonic waves. The ultrasonic waves propagate substantially radially outwardly as indicated by arrows 80 in FIG. 2. Stated another way, cylindrical transducer 20 produces substantially cylindrical wave fronts which propagate generally radially outwardly. These waves are reflected by the interface at active region 32. Because the interface has a parabolic shape, the waves striking any region of the interface will be reflected substantially to focus 44 defined by the surface of revolution, i.e., into a substantially annular or ring-like focal region at focus 44. The ring-like focal region surrounds axis 24 and lies just forward or distal to the forward wall 30 of balloon 28.

The probe 10 includes a bendable section 91 disposed proximal to the first ablation element 11 and thus proximal to the balloons 28 and 50 and ultrasonic transducer 10. The bendable section 91 is controlled by a steering control mechanism 93 so that the bendable section can be selectively bent so as to change the orientation of the first ablation element 11 and the orientation of axis 24. Merely by way of example, the catheter 16 may be provided with one or more pull wires attached to the steering control 93. Other ways of selectively controlling the bending may be used, as for example, pneumatic or hydraulic elements linked to the steering control mechanism.

The features described above may be generally in accordance with the '054 patent and '757 application.

The forward wall 30 of the first balloon 28 is provided with a generally cylindrical extension 35 coaxial with axis 24. Extension 35 desirably is of relatively small diameter, as for example, about 5-20 mm or less, so that the extension can fit within the pulmonary vein. A polymeric sleeve 31 is disposed within extension 35, and extension 35 of the balloon 28 is fastened to the sleeve. A metallic, electrically conductive tubular stiffening element 33 is disposed within the first balloon 28. The stiffening element is mechanically attached to the strain relief barrel 81 and projects distally from the ultrasonic transducer 20. The stiffening element desirably is electrically insulated from the strain relief barrel 81 and ultrasonic transducer 20. The distal end of the stiffening element extends through sleeve 31. An additional ablation element in the form of an electrode 17 is mounted to the stiffening element and sleeve so that the electrode is disposed at the distal extremity of the extension 35 of the first balloon, and the electrode projects slightly beyond the balloon. Thus, the electrode or additional ablation element is disposed distal to the first ablation element 10, and distal to the balloons and ultrasonic transducer. The electrode has a hole or port 95 which communicates with the bore 96 of the stiffening element. The bore 96 of the stiffening element in turn communicates with lumen 18 of catheter 16, so that the lumen 18 and bore 96 cooperatively define a continuous passageway extending from adjacent the proximal end of probe 10 to the distal end of the balloon structure, and communicating with the exterior of the balloon structure on the distal side of the balloon structure.

The stiffening element 33 and electrode 17 are electrically connected to an RF excitation conductor 97 which extends within catheter 16 to adjacent the proximal end of 12 of the probe, where the conductor 97 is electrically connected to an RF excitation source 99. For example, conductor 97 may be a conductor of a coaxial cable.

A sensing element 15 is mounted on the exterior of the device, at or distal to the distal end of the balloon structure 13. For example, sensing element 15 may be a conductive electrode disposed on the exterior of sleeve 31 or on the exterior surface of the extension of the balloon where the extension 35 surrounds the sleeve. The sensing element is connected by one or more conductors (not shown) extending within catheter 16 to a sensing device (not shown) so that the sensing element can be used to detect electrical signals.

In a method according to one embodiment of the invention, the apparatus of FIGS. 1 and 2 can be used to treat atrial fibrillation. With balloons 28 and 50 deflated, the distal end 14 of the probe is advanced into the left atrium of the patient's heart. To facilitate threading, a guide wire may be threaded into the heart and the guide wire may be threaded through the continuous passageway defined by the bore 96 of the stiffening element and the associated lumen 18 of the catheter. Also, the probe may be threaded through one or more sheaths which have previously been threaded into the heart through the vascular system.

With the first ablation element 11 disposed in the left atrium of the heart, the balloons 28 and 50 are inflated with a liquid and gas, respectively. The first ablation element is positioned generally as shown in FIG. 2, with the axis 24 of the first ablation element extending generally perpendicular to the wall 70 of the atrium and with the axis aligned with the ostium of a pulmonary vein 72. As discussed in the '757 publication, the steering arrangement 93 may be used to control the orientation of the axis 24. As also discussed in the '757 publication, the continuous passageway extending through the probe and opening to the distal side of the balloon assembly may be used to introduce a contrast medium through the port 95, so that the contrast medium flows back through the pulmonary vein into the atrium 70. The contrast medium can be used to confirm proper placement of the first ablation element 11.

With the first ablation element in this position, the ring-like focal region 44 is disposed within the heart tissue, near the surface of the heart wall, and encircles the ostium of the pulmonary vein. In this position, the extension 35 of the balloon structure, and the additional ablation element 17 may be disposed within the pulmonary vein or ostium. While the first ablation element is in this position, the ultrasonic transducer 20 is actuated to emit ultrasonic waves. The ultrasonic waves are concentrated in focal region 44. The heart wall tissue located in the focal region is heated rapidly. The rapid heating of the target tissue to the target temperature effectively ablates or kills the tissue at the focal region so that a wall of non-conductive scar tissue forms in the focal region and in neighboring tissue. The time required for ablation will vary with the power applied, but for emitted ultrasonic power on the order of 50 watts, on the order of a few seconds to a few minutes, sonication will form a substantial lesion.

If a complete transmural lesion is formed entirely around the ostium, the tissue within the ostium will be electrically isolated from the remainder of the heart wall. Sensing element 15 may be used to detect electrical signals within the pulmonary vein and ostium, as for example, by moving or steering the probe until the sensing element contacts the wall of the ostium or the wall of the pulmonary vein.

Additional ablation can be performed using the second ablation element 17. For example, if the results of the sensing step indicate that the lesion formed by the first ablation element did not fully block conduction of abnormal electrical signals, additional ablation can be performed at one or more locations on the heart wall so as to complete formation of a ring-like lesion fully encircling an ostium. Alternatively or additionally, the second ablation element can be used to form one or more linear lesions.

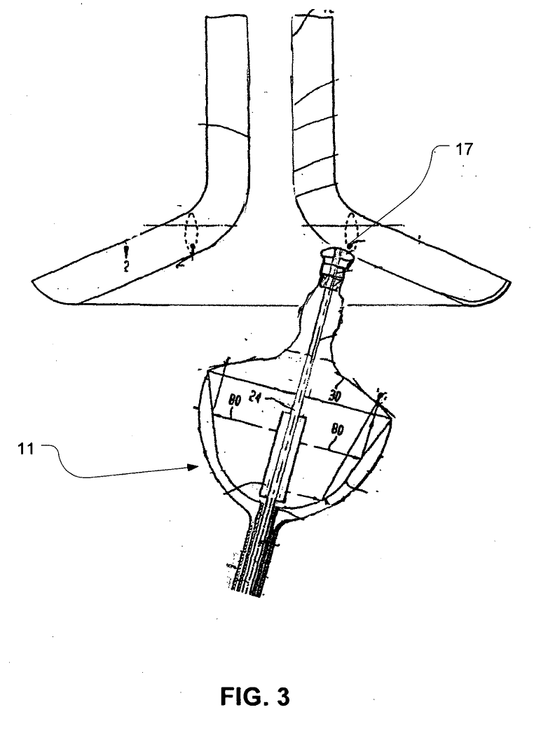

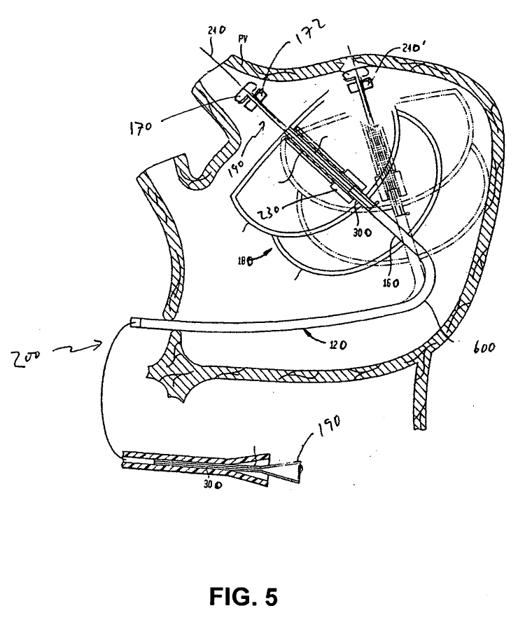

As shown in FIG. 3, the probe is retracted proximally and the second ablation element 17 is positioned at a desired location on the wall of the atrium by using the steering mechanism 93 (FIG. 1) to bend the catheter as needed. With the second ablation element in contact with the heart wall at a location where additional ablation is desired, the RF source 99 (FIG. 1) is actuated to apply RF power to the second ablation element 17. The second ablation element heats tissue in a small spot at and immediately surrounding the point of contact. To form a linear lesion, the second ablation element can be moved continuously or stepwise while repeating the RF actuation.

In this embodiment, the mode of operation of the second ablation element 17 is different from that of the first ablation element 11; the second ablation element 17 ablates the tissue by delivering RF energy to the tissue, whereas the first ablation element ablates using ultrasonic ablation. The ablation device of FIGS. 1-3, therefore, provides two means for ablating tissue. Moreover, the first ablation element 11 is arranged to form a ring-like lesion in each actuation, whereas the second ablation element 17 is arranged to form a localized, spot ablation in each actuation. Both ablation elements are carried into the heart on the same probe, and both can be positioned using the same steering mechanism. Also, as mentioned above, a liquid such as saline solution can be circulated within balloon 28 to cool the ultrasonic transducer. The same circulating liquid also serves to cool electrode 17 of the additional ablation element.

In a variant, the two ablation elements may have the same mode of operation. For example, the RF spot ablation element can be replaced by a spot ultrasonic transducer disposed at the distal end of the balloon structure, i.e., at the location occupied by electrode 17 in the embodiment discussed above.

In a further variant, the sensing element 15 may be omitted. A separate sensing probe may be inserted into through the lumen of the catheter and positioned in the pulmonary vein in the manner described in PCT publication WO 2005/102199, the disclosure of which is hereby incorporated by reference herein.

The stiffening element or tube 33 may be made of steel. However, it is desirable for the stiffening tube 33 to be a good electrical conductor. In one embodiment the stiffening tube is coated with a highly conductive material such as copper, silver, gold or combinations thereof. Such a coating may be in the form of a plated layer or a discrete foil layer covering the outside of the tube. In another embodiment seen in FIG. 4, a distal portion of the stiffening tube 33 is wrapped with a conductive wire 19 to enhance the electrical conduction by the stiffening tube 33. In yet another variant, the stiffening tube 33 is slidable relative to the ultrasonic transducer. For example, the stiffening tube may be arranged to slide proximally relative to the ultrasonic transducer as the balloons are inflated, and may be spring-biased to move distally as the balloons are deflated so as to facilitate collapse of the balloons during deflation. Appropriate flexible or slidable electrical connections between the stiffening tube and the RF conductor in the catheter. In yet another variant, the stiffening tube may be electrically connected to the ultrasonic transducer, as for example, by electrically connecting the stiffening tube to the strain relief barrel 81. In this case, the conductor which transmits electrical excitation signals to the ultrasonic transducer may also carry the RF power to the electrode 17. In a still further variant, the stiffening element may be omitted and the additional ablation element 17 may be supported at the distal end of the balloon assembly constituting the first ablation element. In yet another variant, the port 95 of the distal ablation element may be omitted.

FIG. 6 shows another exemplary embodiment of the ablation device 200. This embodiment includes an insertable structure incorporating an elongated catheter 120 having a proximal end which remains outside of the body, and a distal end 160 adapted for insertion into the body of the subject. The insertable structure also includes a first ablation element 180 mounted to the catheter adjacent distal end 160. Ablation element 180 incorporates a reflector balloon and a structural balloon having a common wall. A cylindrical ultrasonic emitter 230 is mounted within the structural balloon. A lumen 300 is formed within catheter 120. Lumen 300 extends to from the distal end to the proximal end of the catheter 120. As also shown in FIG. 6, positioning of the ablation device 200 within the heart desirably includes selectively controlling the disposition of the forward-to-rearward axis 240 of the device relative to the patient's heart. That is, the position of the forward-to-rearward axis desirably can be controlled by the physician to at least some degree. For example, the device may be arranged so that the physician can selectively reorient the forward-to-rearward axis 240 of the ablation device through a range of motion, as for example, through the range between disposition indicated in solid lines by axis 240 and the disposition indicated in broken lines by axis 2401. To that end, the assembly can be provided with one or more devices for selectively varying the curvature of a bendable region 600 of the catheter just proximal to the ablation device.

In this embodiment, the second or additional ablation element 170 is carried on an additional probe element 190 in the form of an elongated stylet bearing the additional ablation element 170 at or near its distal end. Probe element or stylet 190 may be threaded through the lumen 300 so as to form the assembly shown in FIG. 6. In this assembly, the additional ablation element 170 is also arranged to form a local or spot lesion, whereas the first ablation element 180 is arranged to form a loop. Here again, when the additional probe element 190 and additional ablation element 170 are in place, the additional ablation element 190 and the catheter 120 form a composite probe bearing both the first ablation element 180 and the additional ablation element 170. In this embodiment as well, the additional ablation element 170 may be steered using the same steering mechanism that is used to steer the first ablation element 180. A sensing element 172 may be secured to the second additional probe element 190 proximal to the ablation element 170. The sensing element also will be moved by steering the catheter 120. In this embodiment as well, the ablation element 170 may be a RF transducer or other spot-forming element.

The ablation device of FIG. 6 can be used in a manner similar to the device discussed with reference to FIGS. 1-4. The additional probe element 190 bearing the additional ablation element 170 and sensing element 172 can be assembled with the catheter 120 before or after operating the first ablation element 180. In a further variant, a separate sensing probe can be inserted into the lumen 300 of catheter 120 and then removed and replaced by the additional probe element 190.

As these and other variations and combinations of the features discussed above can be employed, the foregoing description of the preferred embodiments should be taken by way of illustration rather than by way of limitation of the invention.

1. A tissue ablation device comprising:
a catheter;
a first ablation element secured to the catheter; and
an additional ablation element secured to the catheter distal to the first ablation device, the additional ablation element having a mode of operation being different from the ablation device.

2. The tissue ablation device of claim 1, wherein the catheter is adapted to be steered to position at least one of the first ablation element or the additional ablation element in a desired location relative to a tissue to be ablated.

3. The tissue ablation device of claim 1, wherein the first ablation element is capable of making linear or arcuate lesions on the tissue.

4. The tissue ablation device of claim 3, wherein the additional ablation element is adapted to make point lesions on the tissue.

5. The tissue ablation device of claim 4, further comprising an electrically conductive stiffening element extending from the distal end of the catheter, the additional ablation element being mechanically and electrically connected to the stiffening element.

6. The tissue ablation device of claim 5 wherein the stiffening element includes a shaft, at least a portion of the shaft being wrapped with a conductive wire or a conductive foil.

7. The tissue ablation device of claim 5, wherein the stiffening element includes a shaft, at least a portion of the shaft being coated with an electrically conductive layer.

8. The tissue ablation device of claim 3, wherein the first ablation element includes an ultrasonic transducer.

9. The tissue ablation device of claim 7, wherein the additional ablation element includes a RF transducer.

10. The tissue ablation device of claim 8, further comprising:
a sensing element capable of sensing electrical impedance of tissue being ablated.

11. The tissue ablation device of claim 10, wherein the first ablation element includes a balloon structure for focusing ultrasonic energy, the balloon structure surrounding the ultrasonic transducer.

12. The tissue ablation device of claim 11, wherein the additional ablation element is located distal to the balloon structure.

13. An apparatus for cardiac treatment comprising:
a probe having proximal and distal ends;
an ultrasonic first ablation element secured to the probe at or adjacent the distal end thereof, the first ablation element having an expansible balloon structure and an ultrasonic transducer mounted within the balloon structure, the balloon structure having a distal end and a proximal end, the ultrasonic transducer and balloon structure being constructed and arranged so that ultrasonic energy emitted by the ultrasonic transducer will be directed through the balloon structure; and
an additional ablation element secured to the probe and located distal to the ultrasonic transducer and at least partially outside the balloon structure.

14. The apparatus of claim 13, wherein the balloon structure has an axis and wherein the ultrasonic transducer and balloon structure are constructed and arranged to direct ultrasonic energy from the ultrasonic transducer into a ring-like region surrounding the axis.

15. The apparatus of claim 14 wherein the additional ablation element is disposed adjacent the axis of the balloon structure.

16. The apparatus of claim 15 wherein the probe includes a steerable catheter having a bendable section and a steering mechanism for controllably bending the bendable section so as to tilt the axis of the balloon structure and move the additional ablation element.

17. The apparatus of claim 16 wherein the additional ablation element includes an electrode, the apparatus further comprising a stiffening element extending at least partially within the balloon structure, the electrode being electrically connected to the stiffening element, the apparatus further comprising an electrode drive conductor extending within the probe, the electrode drive conductor being electrically connected to the electrode through the stiffening element.

18. The apparatus of claim 17 wherein the stiffening element is tubular and defines a bore, the bore of the stiffening element communicating with the exterior of the balloon structure at or adjacent the distal end of the balloon structure, the probe having a lumen communicating with the bore of the stiffening element.

19. The apparatus of claim 18 wherein the stiffening element is substantially coaxial with the balloon structure.

20. The apparatus of claim 19 wherein the ultrasonic transducer is tubular and substantially coaxial with the balloon structure.

21. The apparatus of claim 16, further comprising a sensing element adapted to sense electrical impedance of the cardiac tissue.

22. The apparatus of claim 13, wherein the probe includes an elongated catheter carrying the ultrasonic first ablation element, the catheter having a lumen communicating with a port at or adjacent the distal end of the balloon structure, the probe further including an additional probe element carrying the additional ablation element, the additional probe element being disposed in the lumen of the catheter.

23. A method of ablating cardiac tissue to impede flow of abnormal electrical signals, the method comprising the steps of:
inserting an elongated probe so that a distal end of the probe and a first ablation element carried on the probe is disposed in a chamber of the heart;
ablating tissue using the first ablation element to form a lesion;
positioning an additional ablation element by steering the probe; and
ablating tissue using the additional ablation element.

24. The method of claim 23, further comprising the step of sensing electrical signals in the tissue to determine whether the lesion will block the abnormal signals before ablating the tissue using the additional ablation element, the step of ablating tissue using the additional ablation element being performed at least in part based on the results of the sensing step.

25. The method of claim 23 further comprising the step of steering the distal end of the probe so as to position the first ablation element relative to the heart before ablating the tissue using the first ablation element.

26. The method of claim 25 wherein the step of ablating the tissue using the first ablation element is performed so as to form a generally loop-like lesion surrounding an axis of the first ablation element, the step of steering the distal end of the probe being performed so as to control the orientation of the axis.

27. The method of claim 26 wherein the additional ablation element is disposed at or near the axis of the first ablation element during the steps of positioning the additional ablation element and ablating tissue using the additional ablation element.

28. The method of claim 26 wherein the step of ablating tissue using the additional ablation element is performed so as to ablate tissue in a point-like region immediately adjacent to the additional ablation element.

29. The method of claim 23, wherein the probe includes an elongated steerable catheter carrying the first ablation element, the method further comprising the step of inserting an additional probe element carrying the additional ablation element into the catheter before the steps of positioning the additional ablation element and ablating tissue using the additional ablation element.

30. The method of claim 23, wherein the first ablation element includes an ultrasonic transducer, the step of ablating tissue using the first ablation element including the step of actuating the ultrasonic transducer to emit ultrasonic energy.

31. The method of claim 23, wherein the additional ablation element includes an electrode, the step of ablating tissue using the additional ablation element including the step of applying RF energy.
